(19) United States
(12) Patent Application Publication
(10) Pub. No.: US 2012/0089128 A1
(43) Pub. Date: Apr. 12, 2012

(54) Anti-Migration Trans-Gastric Jejunal Feeding Tube

(76) Inventors: John A. Rotella, Roswell, GA (US); Donald J. McMichael, Roswell, GA (US); Katherine L. Dziak, Cumming, GA (US)

(21) Appl. No.: 12/899,064

(22) Filed: Oct. 6, 2010

(51) Int. Cl. A61M 39/08

(52) U.S. Cl. 604/529; 604/523

(57) ABSTRACT

The present disclosure describes a trans-gastric jejunal tube (TJ) that avoids migration of the tube upwardly into the stomach. It does this because of alternating stiff and flexible sections. The stiffer sections resist migration and the more flexible or softer portions allow the tube to bend around the pyloris sphincter and into the jejunum. This also allows for a larger diameter feeding lumen than would otherwise be the case.

The present disclosure relates to a trans-gastric feeding tube used in patients that need nutrition delivered directly to the jejunum.

Many patient feeding devices employ a gastrostomy feeding tube. One relatively common situation is to provide nutritional solutions or medicines directly into the stomach or intestines. A stoma is formed in the stomach or intestinal wall and a catheter is placed through the stoma. Feeding solutions can be injected through the catheter to provide nutrients directly to the stomach or intestines (known as enteral feeding). A variety of different catheters intended for enteral feeding have been developed over the years, including some having a “low profile” relative to the portion of the catheter which sits on a patient's skin, as well as those having the more traditional or non-low profile configuration. U.S. Pat. No. 6,019,746 provides an example of such a device.

Enteral feeding may be necessary because of a number of causes, one of is which is the not uncommon reaction following major surgery in which a patient's stomach function is impaired for a period of time. If the patient has a problem with gastric reflux or vomiting, for example, or if the stomach is not adequate for the patient's digestive process requirements, another feeding mode must be chosen. In addition to the need to supply or supplement the body with a certain level of nutrients and the like following surgery as well as in other instances of impaired or limited gastric functionality, a further issue is that an unfed gut can become a source of bacteria that gets into the bloodstream. These types of problems may be resolved by the introduction of nutrients through an enteral feeding device tube properly inserted through the patient's abdominal wall, gastric wall, pylorus, duodenum, and/or into the jejunum beyond the Ligament of Treitz.

Methods of jejunal feeding involve the placing of an extended tube through a stoma in the stomach, past the pyloric sphincter, through the duodenum and into the jejunum. This is a challenging task because of the many twists and turns between the stomach and jejunum. This is a particular challenge because of the sharp bend in the ligament of Treitz between the duodenum and jejunum.

Placement of the tube may use a catheter device that is inserted into the patient through a surgically prepared stoma created in the abdominal wall using traditional surgical procedures. These type of procedures include Stamms Gastrostomy, Witzel Gastrostomy, and others. Interventional radiologists may also place jejunal feeding tubes using fluoroscopy and computed tomography. A growing number of tubes are placed non-surgically, using procedures such as percutaneous gastrostomy. Percutaneous gastrostomy involves the suturing of the stomach to the abdominal wall (gastropexy), and the creation of a stoma using an introducer needle. After the stoma is created it is dilated and the feeding tube may be placed.

While placing a feeding catheter through a stoma in the stomach and into the jejunum has been found to function sufficiently, it has been found that it can be is difficult to maintain the device in place in a stable manner in the patient. The reason for this is the peristaltic action of the muscles of the intestinal tract which can result in the device being moved upwardly into the stomach. Should this occur, it requires replacement of the tube in the jejunum and that involves another surgical procedure with its concomitant risks to the patient.

One attempted solution for this problem has been to use a filler in the jejunal tube to help to stiffen it so that it is less affected by peristaltic action and is more likely to stay where it is placed. While this approach has been effective to a degree, the stiffer trans-jejunal tube requires a larger outer diameter which can cause obstructions and irritation in the bowels or intestines and must also have a smaller diameter internal feeding lumen for the delivery of nutrients, which can easily clog. The larger OD tube is particularly problematic in pediatric applications for obvious reasons.

The stiffer tube is also more difficult to thread from the stomach to the jejunum. The ligament of Treitz, between the duodenum and jejunum, includes a very sharp bend. A stiff tube is quite difficult to thread through this bend and may, particularly for smaller or pediatric patients, distort the anatomy and cause irritation and discomfort.

Alternatively, a tungsten weight at the distal end of the tube has been used in an effort to keep the jejunal tube in place. While this tube is not as stiff, it also has a relatively small feeding lumen which may clog and the weight is often not enough to keep the tube in place. As such, even with the weight, the lack of stiffness or rigidity allows the weighted tube to migrate back upward into the stomach.

Yet another prior art solution has been to insert the feeding tube directly into a stoma in the jejunum. While this is effective in delivering nutrients to the jejunum it involves a different and more involved surgical procedure than those that feed the tube into the stomach and then into the jejunum. This procedure has higher risk of complications for the patient and so is not preferred.

What is needed is a trans-gastric jejunal feeding tube that may be inserted into a stoma in the stomach, extended through the pyloric sphincter, the duodenum, the ligament of Treitz and into the jejunum, and that will remain in place for an extended time without being displaced upwardly back into the stomach.

### SUMMARY

The present disclosure describes a trans-gastric jejunal tube (TJ) that avoids migration of the tube upwardly into the stomach. It does this because of alternating stiff and flexible sections. The stiffer sections resist migration and the more flexible or soft portions allow the tube to bend around the ligament of Treitz and into the jejunum. This also allows for a larger diameter feeding lumen than would otherwise be the case.

The stiffer sections are desirably, through not necessarily, of a greater external diameter than the flexible sections and may have a rounded or tapered end at the transition to the flexible sections. This allows for a user to cut the TJ tube to a desired length at the junction between the stiff and flexible sections. The rounded, curved or tapered end of the stiff sections is desirable so that the end of the tube is less likely to irritate the jejunum. In addition, the sections may be radio-opaque to allow the physician to determine the location of the tube to aid in placement of the tube or to merely monitor its location.

Other objects, advantages and applications of the present disclosure will be made clear by the following detailed description of a preferred embodiment of the disclosure and the accompanying drawings wherein reference numerals refer to like or equivalent structures.

### BRIEF DESCRIPTION OF THE DRAWINGS

FIG. 1 is a view of a trans-gastric jejunal tube showing the flexible and stiff is segments.

FIG. 2 is a cross-sectional view of the area of junction of the flexible and stiff sections, showing the desirably rounded end of the stiff section.

FIG. 3 is a cross-sectional view of the area of junction of the flexible and stiff sections, showing a tapered end on the stiff section.

### DETAILED DESCRIPTION

Reference will now be made to the drawings in which the various elements of the present disclosure will be given numeral designations and in which the disclosure will be discussed so as to enable one skilled in the art to make and use the disclosure. It is to be understood that the following description is only exemplary of the principles of the present disclosure, and should not be viewed as narrowing the pending claims. Those skilled in the art will appreciate that aspects of the various embodiments discussed may be interchanged and modified without departing from the scope and spirit of the disclosure.

In trans-jejunal feeding it is desired to place the distal end or “tail” of the feeding tube in the jejunum where nutrients are desired to be delivered. As described above, the tube is inserted through the stomach, into the duodenum, through the ligament of Treitz and into the jejunum. The ligament of Treitz is particularly challenging because it includes a sharp bend. A very stiff tube will have difficulty in rounding the sharp bend, while an overly flexible tube will be easily displaced upward into the stomach through peristaltic action. The disclosed device answers both of these challenges.

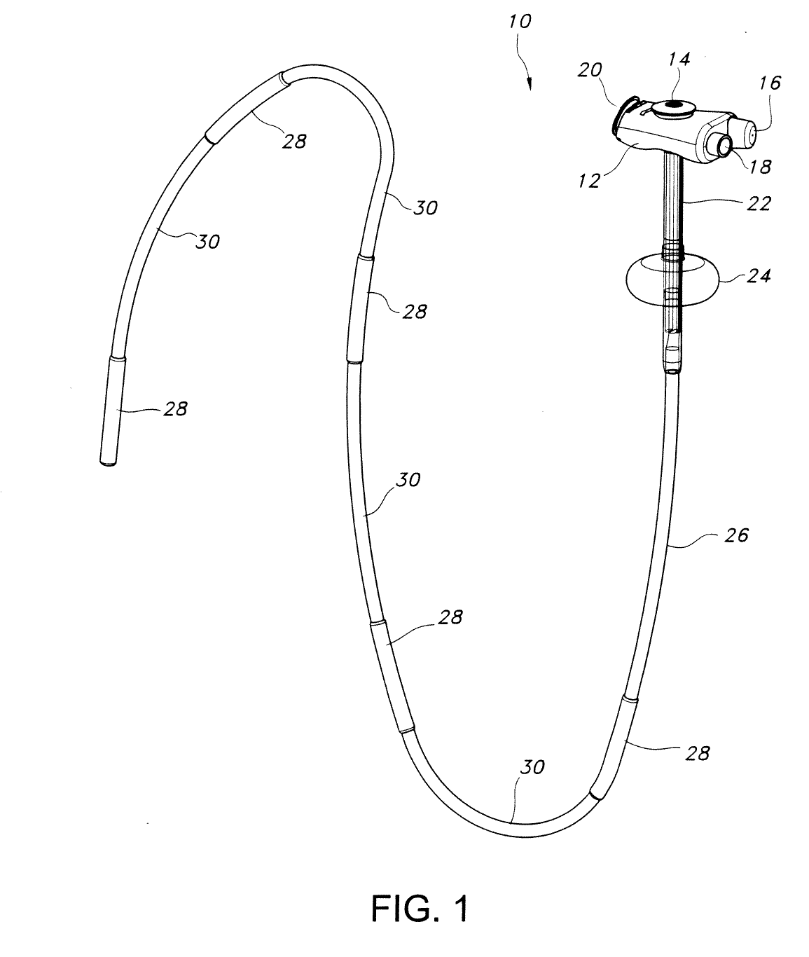

Turning to the drawings, FIG. 1 illustrates an embodiment of the disclosed trans-gastric jejunal (TJ) tube enteral feeding device 10. The device 10 has a base 12 that remains outside the patient's body and through which nutrients are provided to the patient. The base 12 has a proximal side and a distal side and is includes a catheter 22 with a lumen positioned through the base 12. A portion of the catheter 22 extends away from the base 12 on the distal or patient side. A distal end of the catheter of such a device/assembly often includes a balloon 24 which may be expanded to hold the catheter 22 in a position in a body lumen, such as a stomach lumen after, it is installed. It should be noted that the TJ tube 26 disclosed herein may be used with virtually any base, catheter and locking means known in the art, not just those mentioned here.

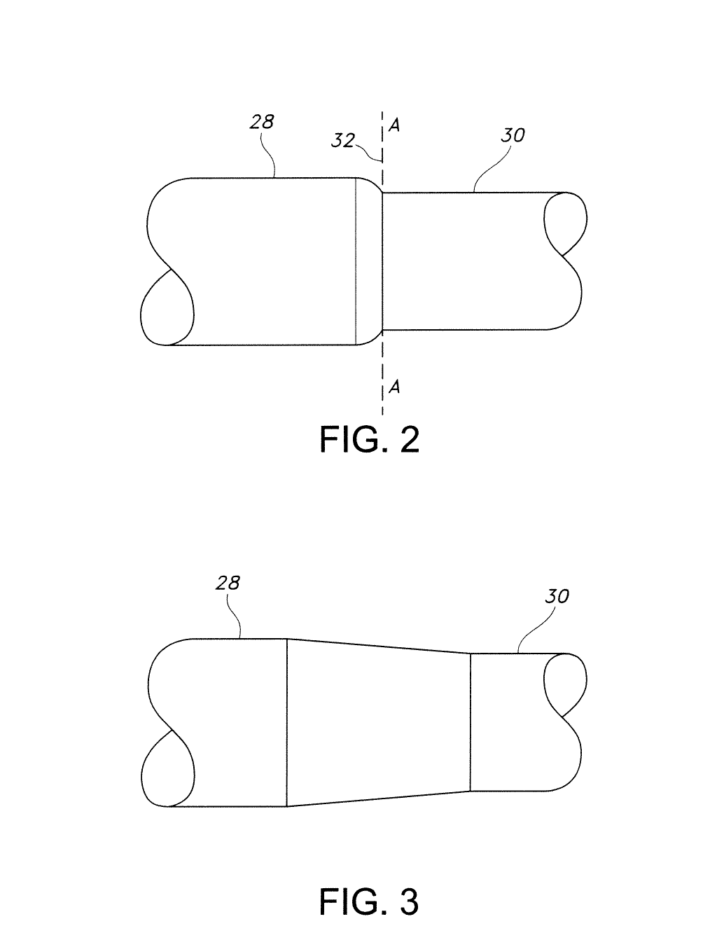

The TJ tube 26 is made of relatively stiff sections 28 and relatively flexible sections 30 alternating along its length as shown in FIG. 1. These stiff and flexible sections 28, 30 may be of the same or different outer diameters. FIG. 2 shows the transition or junction between the two sections and illustrates the desirably curved end 32 of the stiffer sections 28 that may be used when the sections are not the same outer diameter. The curved end helps allow the physician to choose the length desired for the individual patient by cutting the tube 26 at the junction along line A-A. The rounded end 32 should allow for a clean cut between the two sections so that there is no irritation of the jejunal wall from a rough edge.

As noted above, the end of the stiff section may be rounded as shown in FIG. 2, if the stiff and flexible sections have different outer diameters. Alternatively, each stiff section 28 may be tapered as it approaches a flexible section 30 as shown in FIG. 3 so that it transitions smoothly to the flexible section 30.

The alternating stiff and flexible sections may be of varying lengths. The stiff sections may be between 1 and 4 cm long and the flexible sections may be between 5 and 12 cm long. More desirable, the stiff sections may be about 3.5 cm in length and the flexible sections may be about 6.5 cm in length. In another embodiment, the stiff section may be about 3 cm in length and the flexible sections may be about 7 cm in length.

The external diameters of the sections may be the same or may be different from each other, with the stiff sections having an outer diameter between 14 French and 20 French and the flexible sections having an outer diameter between 10 and 16 French. The internal lumen size will vary according to the size of the tube, though it is generally between 7 and 14 French. (Note, French is a measure of circumference based on the theory that non-round tubes of the same circumference will fit into the same incision. One French is approximately 0.33 mm or 0.013 inch).

The disclosed trans-gastric jejunal tube (TJ) avoids migration of the tube upwardly into the stomach through the use of the alternating stiff and flexible sections. The stiffer sections resist upward migration once the tube is in position and the more flexible or soft portions allow the tube to bend around the ligament of Treitz and into the jejunum during placement. It is also believed that the stiffer sections help to reduce reflux into the stomach.

As mentioned above, the tube sections have different softness or hardness such that the flexible sections 30 are relatively more flexible than the stiffer sections 28. The relative hardness of the polymers used to make the sections may be measured by the Shore hardness, a series of scales that is known to those skilled in the art. Hardness is measured using a device called a “durometer”, an instrument specifically developed to measure relative hardness, and is usually performed following ASTM D2240. In the Shore A and D hardness or durometer scales, a higher number indicates a polymer that is harder than a polymer having a lower number within each scale. The Shore A and D scales are used for different types of polymers. Typically the Shore A scale is used for softer, more elastic polymers and the Shore D scale used for stiffer polymers. When comparing the Shore A and Shore D scales, low D values are typically harder than high A values. For example, a 55D hardness is typically harder than a 90A shore hardness value. Desirably, the flexible section of the disclosed tube may have a Shore hardness is between 50A and 70A and the stiffer section may have a Shore hardness between 60A and 90A.

The stiff an flexible sections of the tube are desirably made from the same type of material (though they can be of different hardnesses) as the TJ tube balloon 24 so that joining the components may be easily accomplished. These materials include thermoplastic polyurethane elastomers, thermoplastic polyolefin elastomers, thermoplastic polyolefin block copolymers, SBS di-block elastomers, SEBS tri-block elastomers, polyvinyl chloride, polyethylene terephthalate, silicones and blends and mixtures thereof. A particularly suitable polymer is polyurethane. In one embodiment the stiff sections may be made from Lubrizol's thermoplastic polyurethane elastomer TECOFLEX® EG93A. The flexible sections may be made from TECOFLEX® EG80A. In each case the polymer is a grade of polyurethane but the hardness varies as indicated by the last two numbers and the letter. It should be noted that the stiff and flexible sections may be made from exactly the same polymer, with the difference in flexibility between the two created by differing the thickness of the polymer in the section.

It is also desirable, though not required, that parts of the TJ tube be radio-opaque so that the placement of the tube can be monitored during or after placement. One method of making the tube radio-opaque is through the addition of radio-opaque materials to the polymer from which it is made. Radio-opaque materials are those that absorb and/or block x-rays from passing through an item. These include iodine and barium substances, bismuth salts, tungsten, gold metal, halogenated moieties, metal containing, optically transparent polymers and mixtures thereof.

Halogenated moieties like halogenated diols and halogenated di-isocyanate reactants may be used to prepare polyurethane that is radio-opaque and desirably visually transparent. It has been found that preparing polyurethane using trans cyclo-hexane 1, 4 diisocyanate (t-CHDI) can produce a toxicologically harmless product that is radio-opaque yet visibly transparent. More information on this process may be found in European Patent EP 0 523 928 A2.

Metal containing optically transparent polymers are disclosed in, for example, U.S. Pat. No. 5,856,415 to Lagace et al. and contain a polymer and a metal having a formula (M)((OOC)_{b}R)ₐ where M is a metal atom having an atomic number of at least about 40, R is an organic group selected from aliphatic, cyclo-aliphatic, and aromatic groups containing at least about 3 carbon atoms, b equals the number of carboxyl groups attached to each R group and can be an integer equal to 1 or 2, and a equals the number of organic carboxyl groups (R(OOC)_{b}) attached to each metal (M) atom and is determined by the valence of the metal M, and a is equal to the valence divided by b.

In one embodiment only one type of section of the tube, more likely the stiffer section or sections, may contain a radio-opaque additive, e.g. barium sulfate. In another embodiment, both types of sections of the tube may contain a radio-opaque material which may be different in type and/or amount, resulting in a different degree of radio-opacity for the two sections; e.g. tungsten in the stiffer sections and barium sulfate in the more flexible sections. This differential in radio-opacity allows one to discern the position of the tube using x-rays once it is placed in a patient's jejunum. In yet another embodiment, the type and concentrations of additive (e.g. barium sulfate) may be the same in both types of sections but the sections will appear different under x-ray because of the differing thicknesses of the stiff and flexible sections.

The radio-opaque additive may be present in an amount between 5 and 60 weight percent, more desirably 10 and 40 weight percent or still more desirably between 20 and 30 percent. If a section (e.g. the flexible sections) contains less of the radio-opaque additive than the other section (e.g. the stiffer sections), it may be present in an amount desirably at least 5 weight percent less than the section having more of the additive. The radio-opaque additive may be compounded with the polymeric material from which the tube is made in the conventional manner; e.g., barium sulfate powder is compounded into the polymer through extrusion compounding to produce resin pellets at the proper weight percent addition rate.

The disclosed TJ tube may be produced in a number of ways. The stiff and flexible section could be conventionally extruded as a long tube, the stiff sections injection molded or extruded in sections, and the stiff sections slipped over the more flexible tubing and placed at the proper intervals. The flexible and stiff sections could be produced separately (e.g. injection molded) and heat bonded together at the ends. In another embodiment, the flexible section could be extruded conventionally as a tube and the stiffer sections over-molded onto the flexible tubing at the desired intervals.

As used herein and in the claims, the term “comprising” is inclusive or open-ended and does not exclude additional unrecited elements, compositional components, or method steps.

While various patents have been incorporated herein by reference, to the extent there is any inconsistency between incorporated material and that of the written specification, the written specification shall control. In addition, while the disclosure has been described in detail with respect to specific embodiments thereof, it will be apparent to those skilled in the art that various alterations, modifications and other changes may be made to the disclosure without departing from the spirit and scope of the present disclosure. It is therefore intended that the claims cover all such modifications, alterations and other changes encompassed by the appended claims.

We claim:

1. A trans-gastric jejunal tube (TJ) that avoids migration of said tube upwardly into a stomach comprising alternating stiff and flexible types of sections.

2. The trans-gastric jejunal tube of claim 1 wherein said stiffer sections are of a greater diameter than said flexible sections

3. The trans-gastric jejunal tube of claim 2 wherein said stiffer sections have a rounded end at a transition to the flexible sections.

4. The trans-gastric jejunal tube of claim 2 wherein said stiffer sections have a tapered end at a transition to the flexible sections.

5. The trans-gastric jejunal tube of claim 3 wherein a user may cut said tube to a desired length at the transition between the stiff and flexible sections.

6. The trans-gastric jejunal tube of claim 1 wherein the stiff sections are between 1 and 4 cm long and the flexible sections are between 5 and 12 cm long.

7. The trans-gastric jejunal tube of claim 6 wherein the stiff sections are about 3.5 cm long and the flexible sections are about 6.5 cm long.

8. The trans-gastric jejunal tube of claim 6 wherein the stiff sections are about 3 cm long and the flexible sections are about 7 cm long.

9. The trans-gastric jejunal tube of claim 1 wherein the stiff sections have an outer diameter between 14 and 20 French.

10. The trans-gastric jejunal tube of claim 7 wherein the flexible sections have an outer diameter between 10 and 16 French.

11. The trans-gastric jejunal tube of claim 1 wherein at least one of the types of sections is radio-opaque.

12. The trans-gastric jejunal tube of claim 11 wherein the stiffer sections contain a radio-opaque additive selected from the group consisting of iodine and barium substances, bismuth salts, tungsten, gold metal, halogenated moieties, metal containing, optically transparent polymers and mixtures thereof.

13. The trans-gastric jejunal tube of claim 12 wherein the flexible sections contain a radio-opaque additive selected from the group consisting of iodine and barium substances, bismuth salts, tungsten, gold metal, halogenated moieties, metal containing, optically transparent polymers and mixtures thereof.

14. A trans-gastric jejunal tube (TJ) that avoids migration of said tube upwardly into a stomach, made according to the steps comprising: extruding the flexible sections as a long tube, producing the stiff sections by injection molding or extrusion and slipping the stiff sections over the flexible tube to provide the jejunal tube.

15. A trans-gastric jejunal tube (TJ) that avoids migration of said tube upwardly into a stomach, made according to the steps comprising: extruding the flexible sections as a long tube and over-molding the stiffer sections onto the flexible tube to provide the jejunal tube.
